SEQUENCE LISTING (27 sequences)

SEQ ID NO: 1 - protein, 483 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRYQETSGEYNIQAWTGFNFPGRGTTYSNWKWQWFHFDGTDWDQSRSLSRIFKFHGKAWDWPVSSENGNYDYLMYADYDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGAYDMRNILNNTLVASNPTKAVTLVENHDTQPGQSLESTVQPWFKPLAYAFILTRSGGYPAVFYGDMYGTKGTTTYEIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 2 - DNA, 9515 bp, Artificial Sequence
  synthetic expression cassette
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtcaacttttttgtcttcaccttcaatttctacctgcttaatatctttagcagcttctttataagaactttgaatatcgatcatcttgtccagtgttaagtttgttttgatgtttttttctaatgctgccaaaatctcttgatagtttgttaatgattttaattgtgcaccttcatgaataatttcgttgataatctcacgctgacgctcttgac

SEQ ID NO: 3 - DNA, 1000 bp, Artificial Sequence
  serA1 locus
aatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattc

SEQ ID NO: 4 - DNA, 1578 bp, Bacillus licheniformis
atgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaa

SEQ ID NO: 5 - DNA, 95 bp, Artificial Sequence
  synthetic promoter
gtcgctgataaacagctgacatcaatatcctattttttcaaaaaatattttaaaaagttgttgacttaaaagaagctaaatgttatagtaataaa

SEQ ID NO: 6 - DNA, 58 bp, Bacillus subtilis
acagaatagtcttttaagtaagtctactctgaatttttttaaaaggagagggtaaaga

SEQ ID NO: 7 - DNA, 87 bp, Bacillus licheniformis
atgaaacaacaaaaacggctttacgcccgattgctgacgctgttatttgcgctcatcttcttgctgcctcattctgcagctagcgca

SEQ ID NO: 8 - DNA, 34 bp, Bacillus licheniformis
cggatttcctgaaggaaatccgtttttttatttt

SEQ ID NO: 9 - DNA, 1000 bp, Artificial Sequence
  lysA locus
tcgttccgggttcgatgccgcctccatggatggccatgatgagcatcggcactcttgcgttcttctccgtaattcggtaagaagctggatcttcattccgctccaattcttcaaagcttgaatattgatcgccggactgtgttgcgccatgggccgcagcagaatatgggaatgagatcacagccgaaatcaataaaatattcacaaaaatgtttattgctgtaaaacgatttactgtgttacaatcaacacattgcttcaggcactggagcaatcggtggagtagggatactttggccggtttcatctactccgcctctaaatccaacaaatcagcaaatctcatccttttcaccctggtcattgcctttatcatctcctttgtaaattttttgttaaattcacaatattatatacaattagcccggtcgctgttctatgcatgtcatgatctttttaacaaaaaacacctcccgatcaccgggaagtgtttttcgtgcttgtcattccctctttctctatcgacaaaggtgcaaggggcgctccggttgctgcccgatgcatcttccggcttcatgatagtgcgattcttatgttgtattataacatttcgttttaaaaactaaaccaccatttgtccaaattttcttgatttataaaggaaatgattccctaaacaactaatccattctattgattatagaataacagaaaacagcgcttgatagaaataggcatacacaacgtccattcaataaaaaaagagccgaaaagctgagattttcgcccatacaaataggcatttacctgaatataatcttaaagatgaaaaccaaaaaacgaagtgggaaagaggttatcatatgtatccaaatcgctatccctacacccccgctcccggctatgcacctgaagatgtatacgccccttatcagccggctgcgcattatgaacagagaatttatgagccttatgaagggcagattattacagctccggc

SEQ ID NO: 10 - DNA, 1320 bp, Bacillus licheniformis
ttgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataa

SEQ ID NO: 11 - DNA, 74 bp, Bacillus licheniformis
gcttttcttttggaagaaaatatagggaaaatggtacttgttaaaaattcggaatatttatacaatatcatatg

SEQ ID NO: 12 - DNA, 5822 bp, Artificial Sequence
  expression cassette
aaatataaggaggccaattaaatgatgacagcaaaaaatttgcaagatcgaatcaaattgaataacgggacagctatgccttggtttggacttggcgtatttaaagtagaagaagggcccgaactggtacaagccgtaaaaacggcgattaaacacggctaccgcagtattgataccgcagccatctacggtaatgaagagggggttgggcaaggaatccgcgaggggttgaaagaagccggcatttcaagagaagacctgtttgttacatcaaaggtctggaatgacgatttaggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcatatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagcacacaaggacgcggtacgttcacaatggtctttgaccactatgaagaagtgcctaagagcatcgctgatgagatcatcaaaaaaaatcaaggtgaataattgattttgcctcttcattaaagtataactactattacagaatagtcttttaagtaagtctactctgaatttttttaaaaggagagggtaaagattgaataaatcagtcataccttctttaattacggttggaaactttgtttcgggcatctcttcaatactgcttgcattcagagggtatttattcttagccattatatttgttctgatcggcgctatcttggacagccttgacggcatggccgcgcgcagattaaatgccgtcagcccgtttggaaaagaattagattctttgtctgatattatcactttcggagtcgccccggccatcatcacctattcaatcgttttttatgatgctccgatcttaggccttccttcgacattgttattccctgtttgcggagcgcttcggctggcgcgatttaatattcaatcagagaaccaggattattttacaggccttccgatcaccgcagccggaacgattctggtatgcttaaaccttttctcagaaatcctgggcaaaaagccgttcattttgatcatgctgttcctgtcctttctcatggtcagcaaaatcaaagtcatcagcctgaaggggaaaaaactattgaagaaagtgagctagaagagcagagaggacggatttcctgaaggaaatccgtttttttatttttaacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctcgttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcagattactataacaaccctcaattattgctctctcaattgttcggacataaaaaagggtcttttacaggtgcgggtgaagacaaagcaggattagtcgagcaggcggacgggggcattctgtttatggatgaaatccatcgcctcccgccggaggggcaggaaatgctgttttatttcatagacagcggcacatacaacaggcttggtgaaacagagc

SEQ ID NO: 13 - DNA, 3308 bp, Bacillus licheniformis
atgagcaacactgaacttgatttgctgaggcagcaggcgaatgaattaaacttgcaaatattaaagctgattaacgagcgaggaagaatcgttcaggagattggaaaagcgaaggaagcacaaggcatcaaccgctacgatcctgtcagagaaagagcgatgctgaacgaaattattgaaaataacgacggaccgtttgaaaactcgacgatccagcatatcttcaaagagattttcaaagccgggcttgaactgcaggaagacgatcacagcaaagcgctgctcgtttctcgcaagaaaaagcctgaaaatacaatcgttgatctgaaaggcgaaaaaatcggcgacggagaacaaagatttatcgtcggtccgtgtgcggttgaaagctacgaacaagtagcggaagtcgcagcggcagctaaaaagcaaggcttgaaactgcttcgcggcggagctttcaaaccgcgtacaagcccgtacgacttccaaggcctcggcgtggaaggcctgcaaatcttaaagcgtgttgctgatgagtatgatctggccgtcatcagtgagatcgtgaatccgcagcatattgaagaagccattgattacatcgatgtcatccaaatcggcgcccgcaacatgcagaacttcgagctcttaaaagcggccggttcagtgaagaagccggttctgctgaagcgcggacttgccgcaacgctgaaggaattcatcaatgcagcggagtacatcatgtcgcagggcaatgatcaaatcatcctttgtgaacgcggaatcagaacgtatgaaacagcgactagaaatacgcttgatatctcagctgtgccgatcctgaagcaggaaactcaccttcctgtattcgtggatgtcactcattcaaccggacgccgcgacctgcttcttccgacagcaaaagcagcgcttgcgatcggagctgacggtgtcatggccgaggtccatcctgatccttctgtcgcgctttccgactcagctcagcaaatggatatcccgacatttgaaaaatggttaaatgaactgaagccgctcgttcaagtaaaagcataagatttgtttatggaccgctcgcgagcggtcctttttttacgttgtcatcaaaaattgtcgaaaaaatttcttatttttaagtaaaaagctgtttattcttatttaaattaattataaaaataaagggaacgttttcatattaggtaaaaccgtgtataatttcagagagccccttttgttttggcaggttatgaaaaataatgtaaactggtgttctgtttattttacataaagttttacattttgacatttctctacatgaaaatgtttatgctataggaaaagaaaagtgtacccagttaaggagtggtaaaaatgagtaatgtgacaatatatgatgtagcacgcgaagcaaatgtaagtatggcaaccgtttcccgggtcgtcaacgggaatccgaatgtcaagccgacgacgagaaagaaggtgcttgaagcgatcgagcgtcttggctatcgtccaaacgccgtggcaagagggctggcaagcaaaaagacgacgactgtcggcgtgatcattcccgatatttccagtatcttttattcagagctggcgaggggaatcgaagatatcgcaacgatgtacaagtacaatattattttgagcaattccgaccagaatatggacaaagaacttcatcttttgaatacgatgctaggaaagcaagttgacggtatcgtctttatgagcggaaatgtgactgaagagcatgtggaggaatttaagcgatcgccagttccgatcgtgcttgcggcatctgttgaagaaaaaggggaaacgccgtcggttgcgatcgattatgaacaggcgatttatgatgcggcaaccatgctgattgagaaaggccataagcgccttgcgttcgtctcaggacctatggctgaaccggttaatcaagcgaaaaaactgcaaggctttaaaagagcgcttggggataaggggctgcaatttaaggaagagtatgttgtagaaggcgattatacgtacgattcaggaatggaagcgctggaggctttaatgaagctggatgaaaaaccgacagccgtcctgtcagcgacagacgaaatggcgctcggcgttattcacgcagcacaggataaaggactggctgttccggatgaccttgaagtgatcggttttgacaatacaaggctttcattaatggttcggccgcagctgtcgactgtcgtccagcctacgtatgatatcggtgccgtggcgatgaggcttctgacaaagctgatgaacaaagaggaagtcgaagagcatattgtccagctgccgcatcgcattgaactcagacaatcaacaaaatgatcatcattaaataaacgagaaagaaagcaagtgttcacagcttttttcgtgaattgcttgctttcttttccttgagctggagagattatgaaaaaatgaaacgatttgattatctgacaccggtcggttttattctgggtattttcattttagtgctcggcgttgtctctggagcggggctttccggtttttattcgtttatcgatttgacgtctttttttattgtgacgggtggattgtgtgcagctgtattcatcagcttttccccgaaagatttaaaaagagcgccggctgtgctgaagcaggtctttatttcggaggaagatgatgtgcgcgaattggtcaaaacctttgtcagcctgtctgaacaggcgcgcaaacaaggaattttattattggacgccaatgtcaatgaaatgaaagacccgtttttgaaaaaaggcctgctccttgccatcgacggatgggatgaggagacgatccgcaatgtgatgaactcggaaattgccgcgatggaagaacgacacagaaaggggagaaggatctttgaaaaagcaggagaatttgcgcctgcatggggaatgatcggaacgcttgtcggtctcgtcatgatgctcaaaaacctgaacaagccggaaacgctcggtccgaatatggcaatcgcccttttgacgacgctttacggatcacttttagccaacatgctgtttataccgatcgccgcgaagcttgaagaaaagacggagaatgaaatctttaagaagcaagtcatgatcgaaggcatcatcggcatccagtcgggaagaaatccgcgaaatcttgaaagtcagcttgtcgttttcagttcaaaagaagaatggacaaaaaagcgggcggaccgcatgaaacaaaaggacaggtctcatgaagcttag

SEQ ID NO: 14 - DNA, 924 bp, Bacillus licheniformis
atcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctct

SEQ ID NO: 15 - DNA, 98 bp, Bacillus subtilis
aaaataaccaaaaagcaaggactgctgaaagggctgacataagccttttgccggcggtccttttttaattctgatttttcaaacttagccccctcgag

SEQ ID NO: 16 - DNA, 537 bp, Bacillus licheniformis
ttgaataaatcagtcataccttctttaattacggttggaaactttgtttcgggcatctcttcaatactgcttgcattcagagggtatttattcttagccattatatttgttctgatcggcgctatcttggacagccttgacggcatggccgcgcgcagattaaatgccgtcagcccgtttggaaaagaattagattctttgtctgatattatcactttcggagtcgccccggccatcatcacctattcaatcgttttttatgatgctccgatcttaggccttccttcgacattgttattccctgtttgcggagcgcttcggctggcgcgatttaatattcaatcagagaaccaggattattttacaggccttccgatcaccgcagccggaacgattctggtatgcttaaaccttttctcagaaatcctgggcaaaaagccgttcattttgatcatgctgttcctgtcctttctcatggtcagcaaaatcaaagtcatcagcctgaaggggaaaaaactattgaagaaagtgagctag

SEQ ID NO: 17 - protein, 178 aa, Bacillus licheniformis
LNKSVIPSLITVGNFVSGISSILLAFRGYLFLAIIFVLIGAILDSLDGMAARRLNAVSPFGKELDSLSDIITFGVAPAIITYSIVFYDAPILGLPSTLLFPVCGALRLARFNIQSENQDYFTGLPITAAGTILVCLNLFSEILGKKPFILIMLFLSFLMVSKIKVISLKGKKLLKKVS

SEQ ID NO: 18 - DNA, 136 bp, Bacillus licheniformis
cacacaaggacgcggtacgttcacaatggtctttgaccactatgaagaagtgcctaagagcatcgctgatgagatcatcaaaaaaaatcaaggtgaataattgattttgcctcttcattaaagtataactactatt

SEQ ID NO: 19 - DNA, 227 bp, Bacillus licheniformis
aaatgtgcgcggtccccctgtttcagggggttttttagtttgaaaaaaactttcgctactaaaaaaatttcggccccgtatttttttaaaaaaacataattgaatataaataatacagttaaaactactaagaatctgtaaatagaagtaagctgggccaaaaaataagaaaaaaaaactatagttaaacatttaacaaatgtctgataattgtttataatataaat

SEQ ID NO: 20 - protein, 430 aa, Pseudomonas saccharophila
ADQAGKSPAGVRYHGGDEIILQGFHWNVVREAPYNWYNILRQQASTIAADGFSAIWMPVPWRDFSSWTDGDKSGGGEGYFWHDFNKNGRYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDSWMSDSADSSFCVGELWKEPSEYPPWDWRNTASWQQIIKDWSDRAKCPVFDFALKERMQNGSVADWKHGLNGNPDPRWREVAVTFVDNHDTGYSPGQNGGQHKWPLQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 21 - DNA, 9175 bp, Artificial Sequence
  expression cassette
ctagcgtgtcctcgcatagttcttagattgtcgctacggcatatacgatccgtgagacgtcatcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcagagctcgtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagcttcagcagctgatcaagcaggaaaaagcccggcaggcgtcagatatcatggcggtgatgaaatcatccttcagggctttcattggaacgtcgtcagagaagcgccgtataactggtataacatcctgagacaacaagcgagcacaattgccgctgatggcttttccgcaatctggatgccggttccgtggagagattttagcagctggacggatggagataaaagcggaggcggcgaaggatatttttggcatgactttaacaaaaacggccgctatggaagcgatgctcaactgagacaagcagcaggagcacttggaggagcaggagtcaaagtcctgtacgatgtcgtcccgaaccatatgaaccgcttttatccggacaaagaaatcaatctgccggcaggccaaagattttggagaaacgattgcccggacccgggaaatggaccgaatgattgcgatgatggcgatagatttctgggcggcgaagcggatctgaatacaggccatccgcaaatctatggcatgtttcgggacgaatttacgaatctgagaagcggatatggagcgggcggatttcgctttgattttgtcagaggctatgccccggaaagagttgatagctggatgagcgattcagcggatagcagcttttgcgtcggcgaactttggaaagaaccgagcgaatatccgccgtgggattggagaaatacagcgagctggcagcagatcatcaaagattggagcgatagagcaaaatgcccggtctttgactttgccctgaaagaacgcatgcaaaatggaagcgtcgccgattggaaacatggcctgaacggaaatccggacccgagatggagagaagtcgccgtcacgtttgtcgataaccatgacacaggatatagcccgggacaaaatggaggacaacataaatggccgcttcaagatggccttatcagacaggcgtatgcctatatccttacatcaccgggaacaccggttgtttattggccgcatatgtatgattggggctatggcgatttcatccgccaactgatccaggttagaagaacagcaggagtcagagcggatagcgccattagctttcatagcggctatagcggacttgtcgctacagttagcggcagccaacaaacactggtcgtcgccctgaatagcgatctggcaaatccgggacaagttgctagcggcagctttagcgaagcagtcaatgccagcaatggccaagtcagagtctggagaagcggaagcggagatggaggaggaaatgacggcggctaaaagagcagagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacggttgaacattcttaggctggtcgaatcatttagacacgggcatcgtcctctcgaaaggtg

SEQ ID NO: 22 - protein, 84 aa, Artificial Sequence
  synthetic
FYTFTYFYTFYTFYTFTYFYTFYTFYTFTYFYTFYTFYTFTYFYTFYTFYTFTYFYTFYTFYTFTYFYTFYTFYTFTYFYTFYT

SEQ ID NO: 23 - DNA, 52 bp, Artificial Sequence
  expression cassette
aaaaaaaaaaaaatttttttttttttgggggggggggggccccccccccccc

SEQ ID NO: 24 - DNA, 91 bp, Artificial Sequence
  synthetic promoter
gctgataaacagctgacatcaactaaaagtttcattaaatactttgaaaaaagttgttgacttaaaagaagctaaatgttatagtaattgt

SEQ ID NO: 25 - DNA, 1127 bp, Bacillus licheniformis
catccgaccataatggaacggattcaaaatattgaacagtcttcaactgaatgaagaaaaagctagtgctaaaaacactagctttttctatatgccatttgttagaatatgttattgagcggcagcttcgacattgatcagaccttttccatagtagaaggagcttcccaaataagtcgccgtactggagagacggttgcggacttgtgaagctgaaaggttcggatgttttgacaagatcaaagctgctgctcccgctacatgaggagaagccattgacgttccgttcaatgttgcataagtgctggttgggtaagtgctgtatacgcctgcgccaggagccatgacttcaagctctgctccgacgctggaaaatgaagctctgttgctgttagagtctaccgcaccaacagcgatgacagaatcgtatttcgcaggatagccaattgtattcgtgtttcctgaagatccgctgttccctgctgcagctacaacgacaacccctcttgcatatgcattgtccctatttttttggaggaccaatttaaaattttttggttattaataactgttttagaaaaatgttcagttcgaaagtatgaatagaccgcttcagcctggcagggaaagaggtcccgagggccgcgaatgacaggagattgctccatctgattcattttatgaaaatactattcatttattttatgagatcattttttgccgaaggaggttgcggccttgtcgatcatctattaaaacatgcaatatatcgcatacagaaacgggtgaaagatatgaaatcagctcatgtgaaaggtgtattgatggtgcttgcaggggcttcattgtggggattatcgggaagcgctgcacagtacatatttgaaaggggagcggttgaagccggctcgcttgtctctgtcaggcttctggcatcgggagtcattctcttgttatatgtttcaatgaaaaacggttttcaacatgtgtgtaagatatggaagaaaaaaacagatatttgttcgattttagtattttccatttttggaatgcttgctgttcaatatacgttttttgcttcaatcgaaaagggtaatgccgctgctgctgcaattcttcaatatttg

SEQ ID NO: 26 - DNA, 2472 bp, Bacillus sp.
gcagctaaacccgctgtaagcaacgcttatttagatgcttcaaaccaggtgctggttaaacttagccagccgttaactcttggggaaggcgcaagcggctttacggttcatgacgacacagcaaataaggatattccagtgacatctgtgaaggatgcaagtcttggtcaagatgtaaccgctgttttggcaggtaccttccaacatatttttggaggttccgattgggcacctgataatcacagtactttattaaaaaaggtgactaacaatctctatcaattctcaggagatcttcctgaaggaaactaccaatataaagtggctttaaatgatagctggaataatccgagttacccatctgacaacattaatttaacagtccctgccggcggtgcacacgtcactttttcgtatattccgtccactcatgcagtctatgacacaattaataatcctaatgcggatttacaagtagaaagcggggttaaaacggatctcgtgacggttactctaggggaagatccagatgtgagccatactctgtccattcaaacagatggctatcaggcaaagcaggtgatacctcgtaacgtgcttaattcatcacagtactactattcaggagatgatcttgggaatacctatacacagaaagcaacaacctttaaagtctgggcaccaacttctactcaagtaaatgttcttctttatgacagtgcaacgggttctgtaacaaaaatcgtacctatgacggcatcgggccatggtgtgtgggaagcaacggttaatcaaaaccttgaaaattggtattacatgtatgaggtaacaggccaaggctctacccgaacggctgttgatccttatgcaactgcgattgcaccaaatggaacgagaggcatgattgtggacctggctaaaacagatcctgctggctggaacagtgataaacatattacgccaaagaatatagaagatgaggtcatctatgaaatggatgtccgtgacttttccattgaccctaattcgggtatgaaaaataaagggaagtatttggctcttacagaaaaaggaacaaagggccctgacaacgtaaagacggggatagattccttaaaacaacttgggattactcatgttcagcttatgcctgttttcgcatctaacagtgtcgatgaaactgatccaacccaagataattggggttatgaccctcgcaactatgatgttcctgaagggcagtatgctacaaatgcgaatggtaatgctcgtataaaagagtttaaggaaatggttctttcactccatcgtgaacacattggggttaacatggatgttgtctataatcatacctttgccacgcaaatctctgacttcgataaaattgtaccagaatattattaccgtacggatgatgcaggtaattataccaacggatcaggtactggaaatgaaattgcagccgaaaggccaatggttcaaaaatttattattgattcccttaagtattgggtcaatgagtatcatattgacggcttccgttttgacttaatggcgctgcttggaaaagacacgatgtccaaagctgcctcggagcttcatgctattaatccaggaattgcactttacggtgagccatggacgggtggaacctctgcactgccagatgatcagcttctgacaaaaggagctcaaaaaggcatgggagtagcggtgtttaatgacaatttacgaaacgcgttggacggcaatgtctttgattcttccgctcaaggttttgcgacaggtgcaacaggcttaactgatgcaattaagaatggcgttgaggggagtattaatgactttacctcttcaccaggtgagacaattaactatgtcacaagtcatgataactacaccctttgggacaaaatagccctaagcaatcctaatgattccgaagcggatcggattaaaatggatgaactcgcacaagcagttgttatgacctcacaaggcgttccattcatgcaaggcggggaagaaatgcttcgtacaaaaggcggcaacgacaatagttataatgcaggcgatgcggtcaatgagtttgattggagcaggaaagctcaatatccagatgttttcaactattatagcgggctaatccaccttcgtcttgatcacccagccttccgcatgacgacagctaatgaaatcaatagccacctccaattcctaaatagtccagagaacacagtggcctatgaattaactgatcatgttaataaagacaaatggggaaatatcattgttgtttataacccaaataaaactgtagcaaccatcaatttgccgagcgggaaatgggcaatcaatgctacgagcggtaaggtaggagaatccacccttggtcaagcagagggaagtgtccaagtaccaggtatatctatgatgatccttcatcaagaggtaagcccagaccacggtaaaaag

SEQ ID NO: 27 - DNA, 5913 bp, Artificial Sequence
  expression cassette
aaatataaggaggccaattaaatgatgacagcaaaaaatttgcaagatcgaatcaaattgaataacgggacagctatgccttggtttggacttggcgtatttaaagtagaagaagggcccgaactggtacaagccgtaaaaacggcgattaaacacggctaccgcagtattgataccgcagccatctacggtaatgaagagggggttgggcaaggaatccgcgaggggttgaaagaagccggcatttcaagagaagacctgtttgttacatcaaaggtctggaatgacgatttaggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcatatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagaaatgtgcgcggtccccctgtttcagggggttttttagtttgaaaaaaactttcgctactaaaaaaatttcggccccgtatttttttaaaaaaacataattgaatataaataatacagttaaaactactaagaatctgtaaatagaagtaagctgggccaaaaaataagaaaaaaaaactatagttaaacatttaacaaatgtctgataattgtttataatataaatacagaatagtcttttaagtaagtctactctgaatttttttaaaaggagagggtaaagattgaataaatcagtcataccttctttaattacggttggaaactttgtttcgggcatctcttcaatactgcttgcattcagagggtatttattcttagccattatatttgttctgatcggcgctatcttggacagccttgacggcatggccgcgcgcagattaaatgccgtcagcccgtttggaaaagaattagattctttgtctgatattatcactttcggagtcgccccggccatcatcacctattcaatcgttttttatgatgctccgatcttaggccttccttcgacattgttattccctgtttgcggagcgcttcggctggcgcgatttaatattcaatcagagaaccaggattattttacaggccttccgatcaccgcagccggaacgattctggtatgcttaaaccttttctcagaaatcctgggcaaaaagccgttcattttgatcatgctgttcctgtcctttctcatggtcagcaaaatcaaagtcatcagcctgaaggggaaaaaactattgaagaaagtgagctagaagagcagagaggacggatttcctgaaggaaatccgtttttttatttttaacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctcgttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcagattactataacaaccctcaattattgctctctcaattgttcggacataaaaaagggtcttttacaggtgcgggtgaagacaaagcaggattagtcgagcaggcggacgggggcattctgtttatggatgaaatccatcgcctcccgccggaggggcaggaaatgctgttttatttcatagacagcggcacatacaacaggcttggtgaaacagagc

(19) United States
(12) Patent Application Publication
(10) Pub. No.: US 2024/0263185 A1
(43) Pub. Date: Aug. 8, 2024

(54) COMPOSITIONS AND METHODS FOR ENHANCED PROTEIN PRODUCTION IN BACILLUS CELLS

(71) Applicant: DANISCO US INC., Palo Alto, CA (US)

(72) Inventors: Ryan L. Frisch, Newark, DE (US); Zhen Ma, Garnet Valley, DE (US); Hongxian He, Wilmington, PA (US); Brian Paul, Wilmington, DE (US)

(21) Appl. No.: 18/561,368

(22) PCT Filed: May 23, 2022
(86) PCT No.: PCT/US2022/030521

§ 371 Date: Nov. 16, 2023

Related U.S. Application Data
(60) Provisional application No. 63/192,261, filed on May 24, 2021.

(51) Int. Cl. C12N 15/75; C12N 9/12; C12P 21/02

(52) U.S. Cl. CPC C12N 15/75; C12N 9/1288; C12P 21/02; C12Y 207/08008

(57) ABSTRACT

Certain embodiments of the disclosure are related to recombinant Bacillus strains comprising enhanced protein productivity phenotypes, compositions and methods for constructing such recombinant Bacillus cells, and the like. More particularly, the recombinant Bacillus strains described herein are particularly useful for the enhanced production of proteins of interest when grown/cultivated/fermented under suitable conditions.

### CROSS REFERENCE TO RELATED APPLICATIONS

This application claims benefit to U.S. Provisional Application No. 63/192,261, filed May 24, 2021, which is hereby incorporated by reference in its entirety.

### FIELD

The present disclosure is generally related to the fields of bacteriology, microbiology, genetics, molecular biology, enzymology, industrial protein production the like. Certain embodiments of the disclosure are related to recombinant Bacillus cells (strains) comprising enhanced protein productivity phenotypes, compositions and methods for constructing such recombinant (modified) Bacillus cells, and the like.

### REFERENCE TO A SEQUENCE LISTING

The contents of the electronic submission of the text file Sequence Listing, named “NB41871-US-PSP_SequenceListing.txt” was created on May 13, 2021 and is 64 KB in size, which is hereby incorporated by reference in its entirety.

### BACKGROUND

Gram-positive bacteria such as Bacillus subtilis, Bacillus licheniformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, Bacillus sp. host cells are well known for their production of enzymes (e.g., amylases, cellulases, mannanases, pectate lysases, proteases, pullulanases, etc.) necessary for food, textile, laundry, medical instrument cleaning, pharmaceutical industries and the like. Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority (EFSA), and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) via microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. For example, the expression of many heterologous proteins can still be challenging and unpredictable with respect to yield and the like. As described hereinafter, the present disclosure is related to the highly desirable and unmet needs for obtaining and constructing Bacillus sp, cells (e.g., protein production hosts) having enhanced protein production capabilities.

### SUMMARY

As generally described hereinafter, certain embodiments of the disclosure are related to, among other things, surprising and unexpected results. More particularly, certain embodiments of the disclosure are related to the surprising and unexpected observations that deletion of the wild-type pssA gene (ΔpssA) resulted in decreased production of proteins of interest in Bacillus sp, cells, whereas overexpression of the wild-type prsA gene resulted in increased production of proteins of interest (e.g., enzymes) in such Bacillus cells. As presented and described in the Examples below, the recombinant (genetically modified) Bacillus cells of the instant disclosure are particularly useful for the enhanced production of proteins of interests when cultivated under suitable conditions.

Certain embodiments of the disclosure are therefore related to recombinant (modified) Bacillus cells comprising at least one (one or more) introduced polynucleotide(s) comprising at least 85% sequence identity to the nucleic acid sequence of SEQ ID NO: 16. In related embodiments, the at least one introduced polynucleotide(s) encode a phosphatidylserine synthase (PssA) protein comprising at least 85% sequence identity to SEQ ID NO: 17. For example, in certain embodiments, a recombinant cell may comprise at least one (1) introduced (heterologous) polynucleotide encoding a PssA protein comprising at least 85% sequence identity to SEQ ID NO: 17, and in other embodiments a recombinant cell may comprise at least two (2) introduced (heterologous) polynucleotides encoding PssA proteins comprising at least 85% sequence identity to SEQ ID NO: 17, etc. Thus, in certain embodiments an introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF. In certain preferred embodiments, the recombinant cell produces a protein of interest (POI).

In certain embodiments, a PssA protein comprising at least 85% sequence identity to SEQ ID NO: 17 comprises a conserved PssA superfamily domain. In other embodiments, a PssA protein comprising at least 85% sequence identity to SEQ ID NO: 17 comprises PssA function/activity. In another embodiment, a PssA protein comprising at least 85% sequence identity to SEQ ID NO: 17 comprises a conserved PssA superfamily domain and PssA function/activity.

In certain embodiments, a protein of interest (POI) is an enzyme. In particular embodiments, a protein of interest (POI) includes, but is not limited to, enzymes such as acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhanmo-galacturonases, ribonucleases, transferases, transport proteins, transglutaminases, xylanases and hexose oxidases.

Certain other embodiments are related to recombinant (genetically modified) Bacillus cells derived from parental Bacillus cells producing proteins of interest, wherein the recombinant cells comprise at least one (one or more) introduced polynucleotide(s) encoding a phosphatidylserine synthase (PssA) protein comprising at least 85% sequence identity to SEQ ID NO: 17. In preferred embodiments, the recombinant cells produce increased amounts of the proteins of interest relative to the parental cell (i.e., when grown/cultivated/fermented under the same conditions). In certain related embodiments, the introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF.

Other embodiments relate to recombinant (genetically modified) Bacillus cells derived from parental Bacillus cells comprising a wild-type pssA gene encoding a phosphatidylserine synthase (PssA) protein, wherein the recombinant cells constructed therefrom comprise a genetic modification which replaces the wild-type pssA gene promoter sequence with a heterologous promoter sequence. More particularly, one of skill in the art may obtain parental Bacillus cells comprising a wild-type pssA gene, and genetically modify the cells by knocking-in a heterologous promoter (nucleic acid) sequence to drive and overexpress the pssA gene as desired. In certain related embodiments, a knocked-in heterologous promoter increases pssA gene expression at least 1.25 fold, at least 1.5 fold, at least 1.75 fold, at least 2.0 fold, at least 2.25 fold, at least 2.5 fold, at least 2.75 fold, at least 3.0 fold, at least 5.0 fold, or at least 10.0 fold, relative to the wild-type pssA gene promoter. In other embodiments, the parental cell comprises an introduced expression cassette encoding a protein of interest (POI). In another embodiment, the recombinant cells produce an increased amount of the POI relative to the parental cells (i.e., when grown/cultivated/fermented under the same conditions for the production of the POI).

Certain other embodiments therefore provide (polynucleotide) expression cassettes comprising an upstream (5′) promoter sequence operably linked to a downstream (3′) open reading frame (ORF) encoding a PssA protein comprising at least 85% sequence identity to SEQ ID NO: 17. In certain related embodiments, the cassette further comprises a downstream (3′) terminator sequence operably linked to the upstream (5) ORF.

Certain other embodiments are directed to recombinant Bacillus (host) cells/strains comprising an expression cassette of the instant disclosure.

In yet other embodiments, the disclosure provides methods for producing increased amounts proteins of interest, such methods generally comprising (a) obtaining or constructing a parental Bacillus cell producing one or more proteins of interest and modifying the cell by introducing therein a polynucleotide encoding a phosphatidylserine synthase (PssA) protein comprising at least 85% sequence identity to SEQ ID NO: 17, and (b) cultivating the modified cell under suitable conditions for the production of the one or more proteins of interest, wherein the modified cell produces an increased amount of the one or more proteins of interest relative to the parental cell (i.e., when grown/cultivated/fermented under the same conditions). In certain embodiments of the methods, the introduced polynucleotide is an expression cassette comprising an upstream (5′) promoter sequence operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF. In certain related embodiments, the open reading fame (ORF) sequence encoding the PssA protein comprises at least 85% sequence identity to the nucleic acid sequence of SEQ ID NO: 16. In certain embodiments, a protein of interest is an enzyme, including but not limited to, acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

### BRIEF DESCRIPTION OF THE BIOLOGICAL SEQUENCES

SEQ ID NO: 1 is a nucleic acid (DNA) sequence encoding a Cytophaga sp. α-amylase named “Amylase 1”.

SEQ ID NO: 2 is a synthetic polynucleotide sequence comprising an Amylase 1 expression cassette.

SEQ ID NO: 3 is nucleic acid (DNA) sequence of the B. licheniformis serA1 locus.

SEQ ID NO: 4 is a B. licheniformis serA1 open reading frame (ORF) sequence.

SEQ ID NO: 5 is synthetic p3 promoter nucleic acid sequence.

SEQ ID NO: 6 is a modified B. subtilis aprE 5′ UTR nucleic acid sequence.

SEQ ID NO: 7 is a nucleic acid sequence encoding a B. licheniformis AmyL signal peptide sequence.

SEQ ID NO: 8 is a B. licheniformis amyL transcriptional terminator nucleic acid sequence.

SEQ ID NO: 9 is a nucleic acid sequence of the B. licheniformis lysA locus.

SEQ ID NO: 10 is a B. licheniformis lysA open reading frame (ORF) sequence.

SEQ ID NO: 11 is a B. licheniformis amyL promoter nucleic acid sequence.

SEQ ID NO: 12 is a synthetic polynucleotide sequence comprising pssA expression cassette with tuf promoter.

SEQ ID NO: 13 is a nucleic acid sequence of the B. licheniformis catH locus.

SEQ ID NO: 14 is a synthetic polynucleotide sequence comprising a B. licheniformis catH expression cassette

SEQ ID NO: 15 is B. subtilis spoVG terminator nucleic acid sequence.

SEQ ID NO: 16 is B. licheniformis pssA open reading frame (ORF) sequence encoding a PssA protein of SEQ ID NO: 17.

SEQ ID NO: 17 is the amino acid sequence of the B. licheniformis PssA protein encoded by SEQ ID NO: 16.

SEQ ID NO: 18 is a B. licheniformis tuf promoter nucleic acid sequence.

SEQ ID NO: 19 is a B. licheniformis citZ promoter nucleic acid sequence.

SEQ ID NO: 20 is a nucleic acid sequence encoding a Pseudomonas sacharophia α-amylase named “Amylase 2”.

SEQ ID NO: 21 is a synthetic polynucleotide sequence comprising an Amylase 2 expression cassette.

SEQ ID NO: 22 is a nucleic acid sequence encoding a Pseudomonas sp. α-amylase named “Amylase 3”.

SEQ ID NO: 23 is a synthetic polynucleotide sequence comprising an Amylase 3 expression cassette.

SEQ ID NO: 24 is synthetic p2 promoter nucleic acid sequence.

SEQ ID NO: 25 is a nucleic acid sequence of the B. licheniformis aprL locus.

SEQ ID NO: 26 is a nucleic acid sequence encoding a Bacillus deramificans pullulanase.

SEQ ID NO: 27 is a synthetic polynucleotide sequence comprising pssA expression cassette with citZ promoter.

### DETAILED DESCRIPTION

As described herein, certain embodiments of the disclosure are related to compositions and methods for enhanced protein production in Bacillus sp. (host) cells % strains. More particularly, as set forth hereinafter, and further described in the Examples below, the recombinant (genetically modified) Bacillus cells of the instant disclosure are particularly useful for the enhanced production of proteins of interests when grown/cultivated/fermented under suitable conditions. Thus, certain embodiments of the disclosure are related to, among other things, recombinant polynucleotides (e.g., expression cassettes) encoding phosphatidylserine synthase (PssA) proteins, recombinant Bacillus cells expressing/producing proteins (enzymes) of interest, recombinant Bacillus cells producing proteins of interest and comprising at least one introduced polynucleotide (expression cassette) encoding a PssA protein, compositions and methods for constructing such genetically modified Bacillus cells, method for producing increased amounts proteins of interest and the like.

### I. Definitions

In view of the modified cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”. “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, “the genus Bacillus” includes all species within the genus “Bacillus” as known to those of skill in the art, including but not limited to B. subtilis, B. licheniformis, B, lentus, B. brevis, B. stearothermophilus, B, alkalophilus, B, amyloliquefaciens, B, clausii, B. halodurans, B, megateriun, B. coagulans, B, circudans, B, lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric gene.

As used herein, the term “amylase” refers to a glycoside hydrolase (enzyme) that is, among other things, capable of catalyzing the degradation of starch. Such amylase enzymes include, but are not limited to, endo-acting α-amylases (EC 3.2.1.1: α-D-(1→4)-glucan glucanohydrolase), exo-acting β-amylases (EC 3.2.1.2; α-D-(1→4)-glucan maltohydrolase) and product-specific amylases, such as maltogenic α-amylase (EC 3.2.1.133), α-glucosidases (EC 3.2.1.20; α-D-glucoside glucohydrolase), glucoamylase (EC 3.2.1.3: α-D-(1→4)-glucan glucohydrolase), maltotetraosidases (EC 3.2.1.60), maltohexaosidases (EC 3.2.1.98) and the like.

As used herein, the terms “Amylase 1”. “amylase 1” and/or “amylase 1 protein” refer to a variant Cytophaga sp. α-amylase described in PCT Publication No. WO2014/164777 (incorporated herein by reference in its entirety), wherein the DNA encoding amylase 1 is set forth in SEQ ID NO: 1.

As used herein, the terms “Amylase 2”, “amylase 2” and/or “amylase 2 protein” refer to a variant Pseudomonas sacharophia α-amylase described in PCT Publication No. WO2005/003339 (incorporated herein by reference in its entirety), wherein the DNA encoding amylase 2 is set forth in SEQ ID NO: 20.

As used herein, the terms “Amylase 3”, “amylase 3” and/or “amylase 3 protein” refer to a variant of Pseudomonas sp. α-amylase, which variant amylase 3 was derived from the parental α-amylase described in PCT Publication No. WO2005/003339 (incorporated herein by reference in its entirety).

As used herein, the term “pullulanase” refers to a glycoside hydrolase (enzyme) capable of catalyzing the degradation (debranching) of pullulan, which is a polysaccharide polymer consisting of maltotriose units (α-1,4-glucan;α-1,6-glucan). A pullulanase enzyme (EC 3.2.1.41) may also be referred to as pullulan-6-glucanohydrolase

As used herein, a pullulanase herein named “PULm104” is a truncation of Bacillus deramificans pullulanase described in PCT Publication No. WO99/45124 (incorporated herein by reference in its entirety), wherein the DNA encoding the pullulanase is set forth in SEQ ID NO: 26.

As generally understood by one of skill in the art, such amylases and/or pullulanases are particularly suitable for use in starch liquefaction and saccharification, cleaning starchy stains, textile de-sizing, baking, brewing and the like.

As used herein, a “phosphatidylserine synthase”, abbreviated herein as “PssA”, is among other things, an enzyme which catalyzes a base-exchange reaction in which the polar head group of phosphatidylcholine (PC) or phosphatidylethanolamine (PE) is replaced by L-serine. PssA enzymes are typically classified under enzyme commission (EC) number EC 2.7.8.29, and generally comprise a conserved PssA superfamily domain. For example, in Bacillus sp, cells, the PssA enzyme is responsible for the synthesis of phosphatidylethanolamine (PE), a positively charged phospholipid in the cell membrane.

As used herein, a “wild-type pssA gene” encodes a “native” phosphatidylserine synthase (PssA) protein (i.e., enzyme).

In certain embodiments, a wild-type pssA gene comprises about 80% or greater (nucleotide) sequence identity to SEQ ID NO: 16. In other embodiments, a wild-type pssA gene comprises at least 85%, 86%, 87%, 88%, 89%, 90%, 91%, 92%, 93%, 94%, 95%, 96%, 97%, 98%, 99% or 100% sequence identity to SEQ ID NO: 16.

In certain embodiments, a native PssA enzyme comprises about 85% or greater (amino acid) sequence identity the PssA protein of SEQ ID NO: 17. In other embodiments, a native PssA protein comprises at least 85%, 86%, 87%, 88%, 89%, 90%, 91%, 92%, 93%, 94%, 95%, 96%, 97%, 98%, 99% or 100% sequence identity to SEQ ID NO: 17.

In other embodiments, a wild-type pssA gene comprises at least 85% sequence identity to SEQ ID NO 16, and encodes a functional PssA enzyme comprising at least 85% sequence identity to SEQ ID NO: 17.

As used herein, the “Bacillus cells (strains)” may comprise an endogenous (wild-type) pssA gene encoding a native PssA protein, and as such, when a heterologous (foreign) polynucleotide (e.g., an expression cassette) encoding a functional PssA protein is introduced into a Bacillus cell, the introduced polynucleotide may be referred to herein as a “second (2^{nd}) pssA copy”. In certain embodiments, the heterologous polynucleotide (i.e., 2^{nd} pssA copy) comprises a wild-type pssA gene encoding a native PssA protein. For example, the wild-type pssA gene of SEQ ID NO: 16 encodes a native PssA protein of SEQ ID NO: 17 comprises PssA enzyme activity (function). In other embodiments, the heterologous polynucleotide (i.e., 2^{nd} pssA copy) comprises a nucleic acid sequence encoding a non-native PssA protein. For example, in certain embodiments, a nucleic acid sequence encoding a non-native PssA protein comprises at least about 85% sequence identity to wild-type pssA gene of SEQ ID NO 16. In certain other embodiments, a nucleic acid sequence encoding a non-native PssA protein comprises at least about 85% sequence identity to wild-type pssA gene of SEQ ID NO 16 and encodes a functional (non-native) PssA protein comprising at least 85% to about 99% sequence identity to the native PssA protein of SEQ ID NO: 17. Thus, as described herein, the modified Bacillus cells of the disclosure comprising such introduced heterologous polynucleotide are particularly suitable for expressing native PssA proteins and/or functional PssA variant proteins thereof.

As used herein, a parental B. licheniformis strain named “BF140” or “BF140 (ΔserA1_ΔlysA)” comprises a ser A gene deletion (ΔserA1) and lysA gene deletion (ΔlysA), as described in U.S. Provisional Patent Application No. 62/961,234, filed Jan. 15, 2020 (incorporated herein by reference in its entirety).

As used herein, a B. licheniformis amylase 1 production strain named “BF333” was derived from the (parental) B. licheniformis BF140 strain, wherein the BF333 (daughter) strain comprises two (2) introduced expression cassettes encoding amylase 1.

As used herein, a B. licheniformis (daughter) strain named “ZM1021” was derived from the B. licheniformis (amylase 1) production strain BF333, wherein the ZM1021 strain comprises an introduced expression cassette comprising an upstream (5′) B. licheniformis tuf promoter operably linked to a downstream (3′) pssA ORF.

As used herein, a B. licheniformis (daughter) strain named “ZM1022” was derived from the B. licheniformis (amylase 1) production strain BF333, wherein the ZM1022 strain comprises an introduced expression cassette comprising an upstream (5′) B. licheniformis citZ promoter operably linked to a downstream (3′) pssA ORF.

As used herein, a parental B. licheniformis strain named “LDN0032” comprises a serA gene deletion (ΔserA1) and lysA gene deletion (ΔlysA), as described in U.S. Provisional Patent Application No. 62/961,234, filed Jan. 15, 2020.

As used herein, a B. licheniformis amylase 2 production strain named “LDN253”, was derived from the (parental) B. licheniformis LDN0032 strain, wherein the LDN253 strain comprises two (2) introduced expression cassettes encoding amylase 2.

As used herein, a B. licheniformis (daughter) strain named “ZM1061” was derived from the B. licheniformis (amylase 2) production strain LDN253, wherein the ZM1061 strain comprises an introduced expression cassette comprising an upstream (5′) B. licheniformis tuf promoter operably linked to a downstream (3′) pssA ORF.

As used herein, a B. licheniformis (daughter) strain named “ZM1062” was derived from the B. licheniformis (amylase 2) production strain LDN253, wherein the ZM1062 strain comprises an introduced expression cassette comprising an upstream (5′) B. licheniformis citZ promoter operably linked to a downstream (3′) pssA ORF.

As used herein, a parental B. licheniformis strain named “BF613” comprises a serA gene deletion (ΔserA1) and lysA gene deletion (ΔlysA), as described in U.S. Provisional Patent Application No. 62/961,234, filed Jan. 15, 2020.

As used herein, a B. licheniformis amylase 3 production strain named “WAAA53”, was derived from the (parental) B. licheniformis BF613 strain, wherein the WAAA53 strain comprises two (2) introduced expression cassettes encoding amylase 3.

As used herein, a B. licheniformis (daughter) strain named “WAAA103” was derived from the B. licheniformis (amylase 3) production strain WAAA53, wherein the WAAA103 strain comprises an introduced expression cassette comprising an upstream (5′) B. licheniformis tuf promoter operably linked to a downstream (3′) pssA ORF.

As used herein, a B. licheniformis (daughter) strain named “WAAA104” was derived from the B. licheniformis (amylase 3) production strain WAAA53, wherein the WAAA104 strain comprises an introduced expression cassette comprising an upstream (5′) B. licheniformis citZ promoter operably linked to a downstream (3′) pssA ORF.

As used herein, a parental B. licheniformis strain named “BF144” comprises a deletion of the lysA gene.

As used herein, a B. licheniformis strain named “LDN300” was derived from the parental BF144 strain, wherein the LDN300 strain comprises an introduced expression cassette encoding a truncated pullulanase (PULm104).

As used herein, a B. licheniformis strain named “ZM1134” was derived from the was derived from the B. licheniformis (PULm104) production strain LDN300, wherein the ZM1134 strain comprises an introduced expression cassette comprising an upstream (5′) B. licheniformis tuf promoter operably linked to a downstream (3′) pssA ORF.

As used herein, a B. licheniformis strain named “ZM1135” was derived from the B. licheniformis (PULm104) production strain LDN300, wherein the ZM1135 strain comprises an introduced expression cassette comprising an upstream (5′) B. licheniformis citZ promoter operably linked to a downstream (3′) pssA ORF.

As used herein, a “host cell” refers to a cell that has the capacity to act as a host or expression vehicle for a newly introduced DNA sequence. Thus, in certain embodiments of the disclosure, the host cells are Bacillus sp, or E, coli cells.

As used herein, the phrases a “modified Bacillus cell” and/or a “Bacillus daughter cell” refer to a recombinant Bacillus cell that comprises at least one genetic modification which is not present in the parent Bacillus cell from which the modified Bacillus cell is derived. In certain embodiments, an “unmodified” Bacillus (parent) cell may be referred to as a “control cell”, particularly when being compared with, or relative to, a modified Bacillus cell.

As used herein, when the expression and/or production of a protein of interest (POI) in an “unmodified” (parental) cell is being compared to the expression and/or production of the same POI in a “modified” (daughter) cell, it will be understood that the “unmodified” and “modified” cells are grown/cultivated/fermented under the same conditions (e.g., the same conditions such as media, temperature, pH and the like). In certain embodiments, an increased amount of a protein of interest may be an endogenous Bacillus protein of interest (e.g., native proteases, native amylases, etc.), or a heterologous protein of interest (e.g., recombinant proteases, recombinant amylases, etc.) expressed in a recombinant Bacillus cell of the disclosure.

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, pullulanase activity, cellulase activity, and the like), or total extracellular protein produced as compared to the parental cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof. (b) a gene disruption. (c) a gene conversion. (d) a gene deletion, (e) the down-regulation of a gene, (f) specific mutagenesis and/or (g) random mutagenesis of any one or more the genes disclosed herein.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein. “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence (e.g., an ORF) encoding a protein.

As used herein, “suitable regulatory sequences” refer to nucleotide sequences located upstream (5′ non-coding sequences), within, or downstream (3′ non-coding sequences) of a coding sequence, and which influence the transcription. RNA processing or stability, or translation of the associated coding sequence.

Regulatory sequences may include promoters, translation leader sequences, RNA processing site, effector binding site and stem-loop structure.

As used herein, the term “introducing”, as used in phrases such as “introducing into a bacterial cell” or “introducing into a Bacillus cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or ‘transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the Bacillus sp, chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein. “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb: between 1 bp and 5.0 kb: between 1 bp and 2.5 kb: between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R}. In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (see e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase.

As defined herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell, in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes). PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments. DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a polypeptide of interest that is desired to be expressed in a modified B. licheniformis (daughter) host cell, wherein the POI is preferably expressed at increased levels (i.e., relative to the “unmodified” (parental) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest or an endogenous protein of interest relative to the parental cell. In particular embodiments, an increased amount of a protein of interest produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created.” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison. Wisconsin. USA 53711) (Needleman and Wunsch, 1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein. “specific productivity” is total amount of protein produced per cell per time over a given time period.

As defined herein, the terms “purified” “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

### II. Overexpression of Phosphatidylserine Synthase (PssA) in Bacillus Cells Enhances Protein Production

As generally understood in the art, the cell wall of Bacillus subtilis is a multilayered structure formed by a copolymer of peptidoglycan and anionic polymers (teichoic and teichuronic acid) and contains lipoteichoic acid and proteins. Cao et al. (2017) have described certain aspects of bacterial cell walls that can determine the efficiency of passage by a secretory protein (i.e., the charge density and the crosslinking index of the wall). For example, to study the role of electrostatic interactions between the membrane phospholipids and the secreted protein, Cao et al. (2017) created a library of six (6) engineered B. subtilis strains having modified cell surface components and studied the corresponding influences on protein secretion using α-amylase variants with either low, neutral or high isoelectric points (pI). As concluded in by Cao et al., deletion of the six selected genes (i.e., encoding TagO, TuaA, PssA, ClsA, DacA, or DltA), and the functional consequences on the α-amylase yields suggest that absence (deletion) of phosphatidylserine synthase (PssA) or cardiolipin synthase (ClsA) enhances the α-amylase production, and these beneficial effects can be additive in a double knockout strain (e.g., ΔPssA/ΔClsA).

As generally described herein, and the Examples below. Applicant has constructed recombinant (modified) Bacillus licheniformis cells (strains) expressing a reporter protein of interest (e.g., α-amylase, pullulanase) and a heterologous polynucleotide (cassette) encoding a wild-type phosphatidylserine synthase (PssA) protein. For example, to better understand the PssA enzyme and its role/influence on protein production, three (3) different α-amylase (reporter) proteins (i.e., Examples 1-3; amylases 1-3) and a pullulanase (reporter) protein (Example 4) were assayed for protein production in recombinant B. licheniformis strains comprising the introduced pssA expression cassette (i.e., encoding a 2^{nd} copy of the native PssA protein). As presented in TABLES 1-4, there was an increased amount of reporter protein produced by the recombinant strains comprising the introduced pssA cassette relative to the control strains (i.e., having no introduced pssA expression cassette), which results are surprising in view of the Cao et al. (2017) strains (comprising deletions of the pssA gene).

Thus, as described herein, certain embodiments of the disclosure are related to the surprising and unexpected observation that deletion of the wild-type pssA gene (ΔpssA) resulted in decreased amylase production in Bacillus licheniformis cells (data not shown), whereas overexpression of the wild-type pssA gene resulted in increased amylase and pullulanase production in B. licheniformis cells. More specifically, certain embodiments of the disclosure are related to modified Bacillus cells comprising an introduced polynucleotide encoding a PssA protein comprising at least 85% sequence identity to SEQ ID NO: 17. In particular embodiments, the introduced polynucleotide is an expression cassette comprising an upstream (5′) promoter sequence operably linked to a downstream (3′) open reading frame (ORF) sequence encoding a PssA protein comprising at least 85% sequence identity to SEQ ID NO: 17.

Certain other embodiments are therefore related to modified Bacillus cells derived from parental Bacillus cells producing a protein of interest (POI), wherein the modified cells comprise an introduced polynucleotide encoding a PssA protein comprising at least 85% sequence identity to SEQ ID NO: 17. Thus, certain other embodiments are directed to polynucleotide expression cassettes comprising an upstream (5′) promoter sequence operably linked to a downstream (3′) open reading frame (ORF) sequence encoding a PssA protein of the disclosure. Other embodiments are related to methods for producing an increased amount of a protein of interest (POI) comprising obtaining or constructing a parental Bacillus cell producing a POI and modifying the cell by introducing therein a polynucleotide encoding a PssA protein, and cultivating the modified cell under suitable conditions for the production of the POI, wherein the modified cell produces an increased amount of the POI relative to the parental cell (when cultivated under the same conditions).

### III. Recombinant Polynucleotides and Molecular Biology

As generally described above and hereinafter, certain embodiments are related to recombinant Bacillus cells comprising introduced (heterologous) polynucleotides encoding native PssA proteins. In related embodiments, the recombinant Bacillus cells further comprise introduced (heterologous) polynucleotides encoding one or more proteins of interest (see. Section V). More particularly, as presented below in the Examples, the recombinant polynucleotides, genetically modified Bacillus cells and the like are readily constructed by using routine molecular biology and microbiology techniques and methods know to one skilled in the art. Therefore, the instant disclosure generally relies on routine techniques in the field of recombinant genetics. Basic texts disclosing the general methods of use in present disclosure include Sambrook et al., (2nd Edition, 1989); Kriegler (1990) and Ausubel et al., (1994). Likewise, those of skill in the art are well aware of suitable methods for introducing polynucleotide sequences into bacterial cells (e.g., E, coli, Bacilli, etc.).

Thus, in certain embodiments, a recombinant Bacillus cell comprises an introduced polynucleotide encoding native Bacillus PssA protein comprising an amino acid sequence of SEQ ID NO: 17. In certain other embodiments, a recombinant Bacillus cell comprises an introduced polynucleotide encoding Bacillus PssA protein comprising at least 85% sequence identity to SEQ ID NO: 17. In related embodiments, a recombinant Bacillus cell comprises an introduced polynucleotide encoding PssA protein comprising at least 85% to about 99% sequence identity to SEQ ID NO: 17, wherein the encoded PssA protein comprises a conserved PssA superfamily domain and/or comprises PssA enzyme activity. For example, in certain embodiments, a PssA protein comprising at least 85% to about 99% sequence identity to SEQ ID NO: 17 is transferase enzyme, such as an L-serine-phosphatidylethanolamine phosphatidyltransferase (e.g., Enzyme Commission number EC 2.7.8.29).

Certain other embodiments are therefore related to polynucleotide expression cassettes encoding a PssA protein of the disclosure. For example, in certain embodiments, an expression cassette comprises an upstream (5′) promoter sequence operably linked to a downstream (3′) open reading frame (ORF) sequence encoding a native Bacillus PssA protein comprising an amino acid sequence of SEQ ID NO: 17. In related embodiments, the ORF comprises a nucleotide sequence of SEQ ID NO: 16. In certain other embodiments, the ORF comprises at least 85% to about 99% sequence identity to SEQ ID NO: 16 and encodes a functional PssA protein. Certain other embodiments a related to polynucleotide expression cassettes encoding a protein of interest (POI). Thus, certain other embodiments are related to plasmids, vectors, expression cassettes and the like comprising polynucleotide sequences encoding one or more proteins of the disclosure, recombinant (modified) cells thereof and methods there for constructing such recombinant cells.

Thus, in certain embodiments, a gene, polynucleotide or ORF of the disclosure encoding a Bacillus PssA protein and/or encoding one or more protein of interest is genetically modified, e.g., genetic modifications including, but not limited to, (a) the introduction, substitution, or removal of one or more nucleotides in a gene (or an ORF thereof), or the introduction, substitution, or removal of one or more nucleotides in a regulatory element required for the transcription or translation of the gene or ORF thereof. (b) a gene disruption, (c) a gene conversion. (d) a gene deletion. (e) the down-regulation of a gene. (f) specific mutagenesis and/or (g) random mutagenesis of any one or more the genes disclosed herein.

In particular embodiments, the disclosure relates to recombinant (modified) nucleic acids (polynucleotides) comprising a gene or ORF encoding a native PssA protein (e.g., SEQ ID NO: 17) and/or variant PssA proteins thereof comprising at least 85% to about 99% identity to the PssA of SEQ ID NO: 17 and/or recombinant nucleic acids (polynucleotides) encoding a protein of interest. In certain

Thus, in certain embodiments, a modified Bacillus cell of the disclosure is constructed by increasing the expression of a gene and/or by reducing (or eliminating) the expression of a gene, using methods well known in the art, for example, insertions, disruptions, replacements, or deletions. The portion of the gene to be modified or inactivated may be, for example, the coding region or a regulatory element required for expression of the coding region. An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

Gene deletion techniques enable the partial or complete removal of gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art may readily identify nucleotide regions in the gene's coding sequence and/or the gene's non-coding sequence suitable for complete or partial deletion.

In other embodiments, a modified Bacillus cell of the disclosure is constructed by introducing, substituting, or removing one or more nucleotides in the gene or a regulatory element required for the transcription or translation thereof.

In certain embodiments, a modified Bacillus cell is constructed via CRISPR-Cas9 editing. For example, a wild-type pssA gene encoding a native PssA protein (or functional PssA variant thereof) may be modified via CRISPR-Cas9 editing, by means of nucleic acid guided endonucleases, that find their target DNA by binding either a guide RNA (e.g., Cas9) and Cpfl or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template (e.g., an editing template to replace the native pssA gene promoter sequence with a heterologous promoter). For example, the gene encoding the nucleic acid guided endonuclease (for this purpose Cas9 from S, pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest using Streptococcus pyogenes Cas9, the variable targeting domain (VT) will comprise nucleotides of the target site which are 5′ of the (PAM) proto-spacer adjacent motif (NGG), which nucleotides are fused to DNA encoding the Cas9 endonuclease recognition domain for S. pyrogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500-bp 5′ of targeted gene can be fused to about 500-bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to the cells using many different methods. The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified Bacillus cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been altered. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N-nitro-N-nitrosoguanidine (MNNG). N-methyl-N′-nitrosoguanidine (NTG). O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

International PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences. (3) homologous recombination. (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E, coli and Bacillus sp.). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure are generally chosen so that they are functional in the Bacillus cells, and include, but are not limited to, naturally occurring promoter sequences, synthetic promoter sequences, and/or promoter sequence combinations thereof and the like, which promoter (sequences) are operable/functional in Bacillus cells. Examples of synthetic (engineered) promoters capable of overproducing heterologous (foreign) proteins in Bacillus cells include, but are not limited to, the promoter systems described by Zhou et al. (2019). Wang et al. (2019) and Castillo-Hair et al. (2019). Certain other exemplary Bacillus promoter sequences include, but are not limited to, the B. subtilis alkaline protease (aprE) promoter, the α-amylase promoter of B. subtilis, the α-amylase promoter of B. amyloliquefaciens, the neutral protease (nprE) promoter from B. subtilis, a mutant aprE promoter (e.g., PCT Publication No. WO2001/51643), a B. licheniformis tuf promoter, a B. licheniformis citZ promoter, or any other functional promoter from Bacillus sp, cells. In certain embodiments, a (heterologous) promoter sequence is used to drive the expression of the native PssA protein (or a functional variant thereof), wherein the heterologous promoter increases the expression of the PssA protein at least 1.5 fold relative to the same PssA protein expressed under the control of the wild-type pssA gene promoter. In certain preferred embodiments, the promoter used to drive the expression of a native PssA protein (or a functional variant thereof) increases the expression of the PssA protein at least 1.25 fold, at least 1.5 fold, at least 1.75 fold, at least 2.0 fold, at least 2.25 fold, at least 2.5 fold, at least 2.75 fold, at least 3.0 fold, at least 5.0 fold, or at least 10.0 fold, relative to the expression of the same PssA protein expressed under the control of the wild-type pssA gene promoter. Methods for screening and creating promoter libraries with a range of activities (promoter strength) in Bacillus cells is describe in PCT Publication No. WO2003/089604.

### IV. Fermenting Bacillus Cells for Production of a Protein of Interest

As generally described above, certain embodiments are related to compositions and methods for constructing and obtaining Bacillus cells having increased protein production phenotypes. Thus, certain embodiments are related to methods of producing proteins of interest in Bacillus cells by fermenting the cells in a suitable medium. Fermentation methods well known in the art can be applied to ferment the parental and modified (daughter) Bacillus cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses.

Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the at.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures. e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. Proteins of Interest

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a modified Bacillus cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental) cell.

In certain embodiments, a modified Bacillus cell of the disclosure exhibits an increased specific productivity (Qp) of a POI relative the (unmodified) parental cell. For example, the detection of specific productivity (Qp) is a suitable method for evaluating protein production. The specific productivity (Qp) can be determined using the following equation:

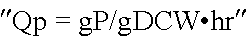

wherein. “gP” is grams of protein produced in the tank; “gDCWV” is grams of dry cell weight (DCV) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1. EC 2, EC 3. EC 4, EC 5 or EC 6.

There are various assays known to those of ordinary skill in the art for detecting and measuring activity of intracellularly and extracellularly expressed proteins.

### VI. Exemplary Embodiments

1. A recombinant (modified) Bacillus cell comprising an introduced polynucleotide comprising at least 85% sequence identity to the nucleic acid sequence of SEQ ID NO: 16.

2. The recombinant cell of embodiment 1, wherein the introduced polynucleotide encodes a phosphatidylserine synthase (PssA) protein comprising at least 85% sequence identity to SEQ ID NO: 17.

3. The recombinant cell of embodiment 1, producing a protein of interest (POI).

4. The recombinant cell of embodiment 1, wherein the introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF.

5. The recombinant cell of embodiment 2, wherein PssA protein comprises a conserved PssA superfamily domain and/or PssA enzyme activity.

6. The recombinant cell of embodiment 3, wherein the POI is an enzyme.

7. A recombinant Bacillus cell derived from a parental Bacillus cell producing a protein of interest (POI), wherein the modified cell comprises an introduced polynucleotide encoding a phosphatidylserine synthase (PssA) protein comprising at least 85% sequence identity to SEQ ID NO: 17.

8. The recombinant cell of embodiment 8, producing an increased amount of the POI relative to the parental cell when cultivated under the same conditions for the production of the POI.

9. The recombinant cell of embodiment 8, wherein the introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3) terminator sequence operably linked to the upstream (5′) ORF.

10. The recombinant cell of embodiment 7, wherein the POI is an enzyme.

11. A recombinant Bacillus cell derived from a parental Bacillus cell comprising a wild-type pssA gene encoding a phosphatidylserine synthase (PssA) protein comprising at least 85% sequence identity to SEQ ID NO: 17, wherein the recombinant cell comprises a genetic modification which replaces the wild-type pssA gene promoter sequence with a heterologous promoter sequence.

12. The recombinant cell of embodiment 11, wherein the heterologous promoter increases pssA gene expression at least 1.5 times relative to the wild-type pssA gene promoter.

13. The recombinant cell of embodiment 11, wherein the parental cell comprises an expression cassette encoding a protein of interest (POI).

14. The recombinant cell of embodiment 13, producing an increased amount of the POI relative to the parental cell when cultivated under the same conditions for the production of the POI.

15. The recombinant cell of embodiment 13, wherein the POI is an enzyme.

16. The recombinant cell of any one of embodiments 6, 10 or 15, wherein the enzyme is selected from the group consisting of acetyl esterases, aminopeptidases, amylases, arabinases, arabinofranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

17. An expression cassette comprising an upstream (5′) promoter sequence operably linked to a downstream (3′) open reading frame (ORF) sequence encoding a PssA protein comprising at least 85% sequence identity to SEQ ID NO: 17, and optionally comprising a downstream (3) terminator sequence operably linked to the upstream (5′) ORF.

18. A recombinant host cell comprising the cassette of embodiment 17.

19. A method for producing an increased amount of a protein of interest (POI) comprising (a) obtaining or constructing a parental Bacillus cell producing a POI and modifying the cell by introducing therein a polynucleotide encoding a phosphatidylserine synthase (PssA) protein comprising at least 85% sequence identity to SEQ ID NO: 17, and (b) cultivating the modified cell under suitable conditions for the production of the POI, wherein the modified cell produces an increased amount of the POI relative to the parental cell when cultivated under the same conditions.

20. The method of embodiment 19, wherein the introduced polynucleotide is an expression cassette comprising an upstream (5′) promoter sequence operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF

21. The method of embodiment 20, wherein the open reading frame (ORF) sequence encoding the PssA protein comprises at least 85% sequence identity to the nucleic acid sequence of SEQ ID NO: 16.

22. The method of embodiment 19, wherein the POI is an enzyme.

23. The method of embodiment 22, wherein the POI is an enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

### EXAMPLES

Certain aspects of the present invention may be further understood in light of the following examples, which should not be construed as limiting. Modifications to materials and methods will be apparent to those skilled in the art. As described herein, all expression cassettes were transformed into the host strains using the methods described PCT Publication No. WO2019/040412 (incorporated herein by referenced in its entirety).

### Example 1

### Enhanced Amylase 1 Production in Bacillus Cells Comprising a PssA Expression Cassette

In the present example, expression cassettes encoding a variant Cytophaga sp. α-amylase (amylase 1) were introduced into B. licheniformis strain BF140 comprising deletions of serA1 and lysA genes. More particularly, a first cassette of amylase 1 (SEQ ID NO: 2) was integrated into the serA1 locus (SEQ ID NO: 3) and contains the serA1 ORF (SEQ ID NO: 4) and the synthetic p3 promoter (SEQ ID NO: 5) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA encoding amylase 1 (SEQ ID NO: 1) operably linked to the B. licheniformis amyL transcriptional terminator (SEQ ID NO: 8). A second cassette of amylase 1 was integrated into the lysA locus (SEQ ID NO: 9) and contains the lysA ORF (SEQ ID NO: 10) and the B. licheniformis amyL promoter (SEQ ID NO: 11) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA encoding amylase 1 (SEQ ID NO: 1) operably linked to the B. licheniformis amyL transcriptional terminator (SEQ ID NO: 8). This resulted in the amylase 1 production strain herein named “BF333”.

A pssA expression cassette comprising SEQ ID NO: 12 or SEQ ID NO: 27 was then integrated at the catH locus (SEQ ID NO: 13) of the amylase 1 production strain BF333. More particularly, the pssA expression cassettes contain the native B. licheniformis catH expression cassette (SEQ ID NO: 14) operably linked to the B. subtilis spoVG transcription terminator (SEQ ID NO: 15) operably linked to a promoter operably linked to the modified B. subtilis aprE 5′ UTR (SEQ ID NO: 6) operably linked to the B. licheniformis pssA ORF (SEQ ID NO: 16) operably linked to the B. licheniformis amyL transcriptional terminator (SEQ ID NO: 8). In the instant example, B. licheniformis tuf (SEQ ID NO: 18) and citZ (SEQ ID NO: 19) promoters were used to drive pssA expression (i.e., expression cassettes SEQ ID NO: 12 and SEQ ID NO: 27, respectively), and resulted in amylase 1 production strains named “ZM1021” and “ZM1022”, respectively.

The three (3) amylase 1 production strains (BF333, ZM1021, ZM1022) were assayed for production of α-amylase using standard small scale conditions (as described in PCT publication No. WO2018/156705 and WO20191055261, each incorporated herein by reference). The α-amylase produced was quantified using the method of Bradford or the Ceralpha assay. The relative improvement in amylase production strains comprising the introduced pssA expression cassette was compared to the parent strain BF333, as presented below in TABLE 1. The results shown in TABLE 1 demonstrate an improvement of amylase production in the strains comprising a second (2^{nd}) copy of the native pssA gene controlled by a heterologous promoter (e.g., either tuf or citZ promoter).

TABLE 1

| | | Relative |
|---|---|---|
| | | Amylase 1 |
| Strain | Description | Production |
| BF333 | 2 copies Amylase 1; WT pssA gene | 1.00 |
| ZM1021 | 2 copies Amylase 1; WT pssA gene + pssA | 1.08 |
| | cassette (tuf) | |
| ZM1022 | 2 copies Amylase 1; WT pssA gene + pssA | 1.19 |
| | cassette (citZ) | |

### Relative Expression of Amylase 1 in Strains Containing a PssA Expression Cassette

### Example 2

### Enhanced Amylase 2 Production in Bacillus Cells Comprising a PssA Expression Cassette

In the instant example, amylase 2 expression cassettes were introduced into B. licheniformis strain LDN0032 comprising deletions of both serA1 and lysA genes, as generally described above in Example 1. More particularly, a first cassette of amylase 2 (SEQ ID NO: 21) was integrated into the lysA locus (SEQ ID NO: 9) and contains the lysA ORF (SEQ ID NO: 10) and the synthetic p3 promoter (SEQ ID NO: 5) operably linked to the modified B. subtilis aprE 5′ UTR (SEQ ID NO: 6) operably linked to the DNA encoding B. licheniformis AmyL signal peptide (SEQ ID NO: 7) sequence operably linked to the DNA encoding amylase 2 (SEQ ID NO: 20) operably linked to the B. licheniformis amyL transcriptional terminator (SEQ ID NO: 8). A second cassette of amylase 2 was integrated into the serA1 locus (SEQ ID NO: 3) and contains the B. licheniformis amyL promoter (SEQ ID NO: 11) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA encoding amylase 2 (SEQ ID NO: 20) operably linked to the B. licheniformis amyL transcriptional terminator (SEQ ID NO: 8) operably linked to the serA1 ORF (SEQ ID NO: 4). This resulted in the amylase 2 production strain herein named “LDN253”.

A pssA expression cassette comprising SEQ ID NO: 12 or SEQ ID NO: 27 was then integrated at the catH locus (SEQ ID NO: 13) of the amylase 2 production strain LDN253. The pssA expression cassettes contain the native B. licheniformis catH expression cassette (SEQ ID NO: 14) operably linked to the B. subtilis spoVG transcription terminator (SEQ ID NO: 15) operably linked to a promoter operably linked to the modified B. subtilis aprE 5′ UTR (SEQ ID NO: 6) operably linked to the B. licheniformis pssA ORF (SEQ ID NO: 16) operably linked to the B. licheniformis amyL transcriptional terminator (SEQ ID NO: 8). In the present example, B. licheniformis tuf (SEQ ID NO: 18) and citZ (SEQ ID NO: 19) promoters were used to drive pssA expression (i.e., expression cassettes SEQ ID NO: 12 and SEQ ID NO: 27, respectively), and resulted in amylase 2 production strains named “ZM1061” and “ZM1062”, respectively.

The three (3) amylase 2 production strains (LDN253, ZM1061, ZM1062) were assayed for production of α-amylase using standard small scale conditions (as described in PCT publication No. WO2018/156705 and WO2019/055261). The amylase 2 produced was quantified using the method of Bradford or the Ceralpha assay. The relative improvement in amylase production strains comprising the introduced pssA expression cassette was compared to the parent strain LDN253, as presented below in TABLE 2. The results shown in TABLE 2 demonstrate an improvement of amylase production in strains comprising a second (2^{nd}) copy of the native pssA gene controlled by either tuf or citZ promoter.

TABLE 2

| | | Relative |
|---|---|---|
| | | Amylase 2 |
| Strain | Description | Production |
| LDN253 | 2 copies Amylase 2; WT pssA gene | 1.00 |
| ZM1061 | 2 copies Amylase 2; WT pssA gene + pssA | 1.11 |
| | cassette (tuf) | |
| ZM1062 | 2 copies Amylase 2; WT pssA gene + pssA | 1.12 |
| | cassette (citZ) | |

### Relative Expression of Amylase 2 in Strains Containing a PssA Expression Cassette

### Example 3

### Enhanced Amylase 3 Production in Bacillus Cells Comprising a PssA Expression Cassette

In the instant example, amylase 3 expression cassettes were introduced into B. licheniformis strain BF613 comprising deletions of both serA1 and lysA genes, as generally described above in Example 1. More particularly, a first cassette of amylase 3 (SEQ ID NO: 23) was integrated into the serA1 locus (SEQ ID NO: 3) and contains the serA1 ORF (SEQ ID NO: 4) and the synthetic p3 promoter (SEQ ID NO: 5) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA encoding amylase 3 (SEQ ID NO: 22) operably linked to the B. licheniformis amyL transcriptional terminator (SEQ ID NO: 8). A second cassette of amylase 3 was integrated into the lysA locus (SEQ ID NO: 9) and contains the lysA ORF (SEQ ID NO: 10) and the synthetic p2 promoter (SEQ ID NO: 24) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA encoding amylase 3 (SEQ ID NO: 22) operably linked to the B. licheniformis amyL transcriptional terminator (SEQ ID NO: 8). This resulted in amylase 3 production strain herein named “WAAA53”.

A pssA expression cassette comprising SEQ ID NO: 12 or SEQ ID NO: 27 was then integrated at the aprL locus (SEQ ID NO: 25) of the amylase 3 production strain WAAA53. The pssA expression cassettes contain the native B. licheniformis catH expression cassette (SEQ ID NO: 14) operably linked to the B. subtilis spoVG transcription terminator (SEQ ID NO: 15) operably linked to a promoter operably linked to the modified B. subtilis aprE 5′ UTR (SEQ ID NO: 6) operably linked to the DNA encoding B. licheniformis pssA ORF (SEQ ID NO: 16) operably linked to the B. licheniformis amyL transcriptional terminator (SEQ ID NO: 8). In the present example, B. licheniformis tuf (SEQ ID NO: 18) and citZ (SEQ ID NO: 19) promoters were used to drive pssA expression (i.e., expression cassettes SEQ ID NO: 12 and SEQ ID NO: 27, respectively), and resulted in amylase 3 production strains “WAAA103” and “WAAA104”, respectively.

The three (3) amylase 3 production strains (WAAA53. WAAA103, WAAA104) were assayed for production of α-amylase using standard small scale conditions (as described in PCT publication No. WO2018/156705 and WO2019/055261). The amylase 3 produced was quantified using the method of Bradford or the Ceralpha assay. The relative improvement in amylase production strains comprising the introduced pssA expression cassette was compared to the parent strain WAAA53, as presented below in TABLE 3. The results shown in TABLE 3 demonstrate an improvement of amylase production in strains comprising a second (2^{nd}) copy of the native pssA gene controlled by either tuf or citZ promoter.

TABLE 3
RELATIVE EXPRESSION OF AMYLASE 3 IN STRAINS
CONTAINING A PSSA EXPRESSION CASSETTE

| | | Relative |
|---|---|---|
| | | Amylase 3 |
| Strain | Description | Production |
| WAAA53 | 2 copies Amylase 3; WT pssA gene | 1.00 |
| WAAA103 | 2 copies Amylase 3; WT pssA gene + pssA | 1.06 |
| | cassette (tuf) | |
| WAAA104 | 2 copies Amylase 3; WT pssA gene + pssA | 1.07 |
| | cassette (citZ) | |

### Example 4

### Enhanced Pullulanase Production in Bacillus Cells Comprising a PssA Expression Cassette

In the present example, a pullulanase expression cassette was introduced into B. licheniformis strain BF144 comprising a deletion of lysA gene. More particularly, the expression cassette contains the lysA ORF (SEQ ID NO: 10) and the synthetic p3 promoter (SEQ ID NO: 5) operably linked to the modified B. subtilis aprE 5′ UTR (SEQ ID NO: 6) operably linked to the DNA encoding B. licheniformis AmyL signal peptide sequence (SEQ ID NO: 7) operably linked to the DNA (SEQ ID NO: 26) encoding the pullulanase enzyme operably linked to the B. licheniformis amyL transcriptional terminator (SEQ ID NO: 8). This resulted in pullulanase production strain herein named “LDN300”.

A pssA expression cassette comprising SEQ ID NO: 12 or SEQ ID NO: 27 was then integrated at the catH locus (SEQ ID NO: 13) of the pullulanase production strain LDN300. The pssA expression cassettes contain the native B. licheniformis catH expression cassette (SEQ ID NO: 14) operably linked to the B. subtilis spoVG transcription terminator (SEQ ID NO: 15) operably linked to a promoter operably linked to the modified B. subtilis aprE 5′ UTR (SEQ ID NO: 6) operably linked to the DNA encoding B. licheniformis pssA ORF (SEQ ID NO: 16) operably linked to the B. licheniformis amyL transcriptional terminator (SEQ ID NO: 8). In the present example, B. licheniformis tuf (SEQ ID NO: 18) and citZ (SEQ ID NO: 19) promoters were used to drive pssA expression (i.e., expression cassettes SEQ ID NO: 12 and SEQ ID NO: 27, respectively), and resulted in pullulanase production strains “ZM1134” and “ZM1135”, respectively.

The three (3) pullulanase production strains (LDN300. ZM1134, ZM1135) were assayed for production of pullulanase using standard small scale conditions (as described in PCT publication No. WO2018/156705 and WO2019/055261). Pullulanase was quantified using the method of Bradford assay. The relative improvement in pullulanase production strains containing an introduced pssA expression cassette was compared to the parent strain LDN300, as presented below in TABLE 4. The results shown in TABLE 4 demonstrate an improvement of pullulanase production in strains comprising a second (2^{nd}) copy of the native pssA gene controlled by either tuf or citZ promoter.

TABLE 4
RELATIVE EXPRESSION OF PULLULANASE IN STRAINS
CONTAINING A PSSA EXPRESSION CASSETTE

| | | Relative |
|---|---|---|
| | | Pullulanase |
| Strain | Description | Production |
| LDN300 | 1 copy Pullulanase; WT pssA gene | 1.00 |
| ZM1134 | 1 copy Pullulanase; WT pssA gene + pssA | 1.05 |
| | cassette (tuf) | |
| ZM1135 | 1 copy Pullulanase; WT pssA gene + pssA | 1.09 |
| | cassette (citZ) | |

### REFERENCES

PCT Publication No. WO2001/51643
PCT Publication No. WO2002/14490
PCT Publication No. WO2003/083125
PCT Publication No. WO2005/003339
PCT Publication No. WO2014/164777
PCT Publication No. WO2018/156705
PCT Publication No. WO2018/156705
PCT Publication No. WO2019/040412
PCT Publication No. WO2019/055261
PCT Publication No. WO2019/055261
PCT Publication No. WO099/45124
U.S. Provisional Patent Application No. 62/961,234
Albertini and Galizzi, Bacteriol., 162:1203-1211, 1985.
Ausubel et al., (1994)
Cao et al., “Cell surface engineering of Bacillus subtilis improves production yields of heterologously expressed α-amylases”, Microb. Cell Fact., 16:56, 2017.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Castillo-Hair et al., “An Engineered B. subtilis Inducible Promoter System with over 10 000-Fold Dynamic Range”, ACS Synth. Biol., 8(7): 1673-1678, 2019.
Earl et al., “Ecology and genomics of Bacillus subtilis”, Trends in Microbiology., 16(6):269-275, 2008.
Ferrari et al., “Genetics,” in Hartwood et al. (ed.), Bacillus, Plenum Publishing Corp., 1989.
Matsumoto. “Phosphatidylserine synthase from bacteria”, Review Biochim Biophys Acta, 1348(1-2):214-227, 1997.
Olempska-Beer et al., “Food-processing enzymes from recombinant microorganisms—a review” Regul. Toxicol. Pharmacol., 45(2):144-158, 2006.
Sambrook. J.; Fritsch, E. F.; Maniatis, T. “Molecular cloning: a laboratory manual”, 1989 2^{nd} Ed.; pp.xxxviii+1546 pp.

### Stahl and Ferrari, J. Bacteriol., 158:411-418, 1984.

Van Dijl and Hecker, “Bacillus subtilis: from soil bacterium to super-secreting cell factory”, Microbial Cell Factories, 12(3). 2013.
Wang et al., “Engineering strong and stress-responsive promoters in Bacillus subtilis by interlocking sigma factor binding motifs”. Synth. Syst. Biotechnol., 4(4): 197-203, 2019.
Zhou et al., “Promoter engineering enables overproduction of foreign proteins from a single copy expression cassette in Bacillus subtilis”, Microbial Cell Factories, 18011), 2019.

1. A recombinant Bacillus sp, cell comprising an introduced polynucleotide comprising at least 85% sequence identity to the nucleic acid sequence of SEQ ID NO: 16.

2. The recombinant cell of claim 1, wherein the introduced polynucleotide encodes a phosphatidylserine synthase (PssA) protein comprising at least 85% sequence identity to SEQ ID NO: 17.

3. The recombinant cell of claim 1, producing a protein of interest (POI).

4. The recombinant cell of claim 1, wherein the introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF.

5. The recombinant cell of claim 3, wherein the POI is an enzyme.

6. A recombinant Bacillus sp, cell derived from a parental Bacillus sp, cell producing a protein of interest (POI), wherein the recombinant cell comprises an introduced polynucleotide encoding a phosphatidylserine synthase (PssA) protein comprising at least 85% sequence identity to SEQ ID NO: 17 and produces an increased amount of the POI relative to the parental cell when cultivated under the same conditions.

7. The recombinant cell of claim 6, wherein the introduced polynucleotide is an expression cassette comprising an upstream (5′) promoter operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF.

8. The recombinant cell of claim 6, wherein the POI is an enzyme.

9. A recombinant Bacillus sp, cell derived from a parental Bacillus sp, cell comprising a wild-type pssA gene encoding a native phosphatidylserine synthase (PssA) protein, wherein the recombinant cell comprises an introduced heterologous promoter sequence which replaces the wild-type pssA gene promoter sequence of the parental cell.

10. The recombinant cell of claim 9, wherein the heterologous promoter increases pssA gene expression at least 1.2 fold relative to the wild-type pssA gene promoter.

11. The recombinant cell of claim 9, wherein the parental cell comprises an expression cassette encoding a protein of interest (POI).

12. The recombinant cell of claim 11, producing an increased amount of the POI relative to the parental cell when cultivated under the same conditions.

13. The recombinant cell of claim 11, wherein the POI is an enzyme.

14. (canceled)

15. An expression cassette comprising an upstream (5′) promoter sequence operably linked to a downstream (3′) open reading frame (ORF) sequence encoding a PssA protein comprising at least 85% sequence identity to SEQ ID NO: 17, and optionally comprising a downstream (3′) terminator sequence operably linked to the upstream (5′) ORF.

16. (canceled)

17. A method for producing an increased amount of a protein of interest (POI) comprising:
(a) obtaining or constructing a parental Bacillus cell producing a POI and modifying the cell by introducing therein a polynucleotide encoding a phosphatidylserine synthase (PssA) protein comprising at least 85% sequence identity to SEQ ID NO: 17, and
(b) cultivating the modified cell under suitable conditions for the production of the POI,
wherein the modified cell produces an increased amount of the POI relative to the parental cell when cultivated under the same conditions.

18. The method of claim 17, wherein the introduced polynucleotide is an expression cassette comprising an upstream (5′) promoter sequence operably linked to a downstream (3′) open reading frame (ORF) encoding a PssA protein comprising at least 85% sequence identity to SEQ ID NO: 17, and optionally comprising a downstream (3′) terminator sequence operably linked to the upstream (5′) ORF

19. The method of claim 18, wherein the open reading frame (ORF) sequence encoding the PssA protein comprises at least 85% sequence identity to the nucleic acid sequence of SEQ ID NO: 16.

20. The method of claim 17, wherein the POI is an enzyme.

21. The method of claim 20, wherein the POI is an enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.
